(19) United States
(12) Patent Application Publication
(10) Pub. No.: US 2007/0038068 A1
(43) Pub. Date: Feb. 15, 2007

(54) Mr imaging method

(76) Inventors: Christoph Gunther Leussler, Hamburg (DE); Peter Bornert, Hamburg (DE)

Correspondence Address: PHILIPS INTELLECTUAL PROPERTY & STANDARDS, 595 MINER ROAD, CLEVELAND, OH, 44143, US

(21) Appl. No.: 10/548,986

(22) PCT Filed: Feb. 27, 2004
(86) PCT No.: PCT/IB04/50164

§ 371 Date: Jul. 7, 2006

(30) Foreign Application Priority Data
Mar. 10, 2003 (EP) 03100586.1

(51) Int. Cl. A61B 5/05

(52) U.S. Cl. 600/410

(57) ABSTRACT

The invention relates to an MR method for generating an image of a body part of a patient located in the examination volume of an MR device. According to the method, firstly MR signals are excited in the examination volume by means of a sequence of magnetic field gradient pulses (15, 16, 17) and/or RF pulses (14). Thereafter, the MR signals are recorded by means of an RF coil arrangement of the MR device, where the RF coil arrangement has a number of coil elements that can be actuated individually. Finally, image reconstruction from the recorded MR signals takes place. For the purpose of imaging that is as rapid as possible while at the same time physiologically exposing the patient to magnetic field gradient pulses or RF pulses to a minimum extent, the invention proposes that the spatial RF field distribution during excitation of the MR signals and/or the spatial sensitivity profile of the RF coil arrangement during recording of the MR signals be varied by suitable actuation (C 1,C2) of the RF coil arrangement.

The invention relates to an MR method for generating an image of a body part of a patient located in the examination volume of an MR device, comprising the following method steps:
a) excitation of MR signals in the examination volume by means of a sequence of magnetic field gradient pulses and/or RF pulses,
b) recording of the MR signals by means of an RF coil arrangement of the MR device,
c) image reconstruction from the recorded MR signals.

In addition, the invention relates to an MR device for carrying out the method and to a computer program for such an MR device.

In MR (magnetic resonance) imaging, nuclear magnetization within the examination volume is usually located by means of magnetic fields (magnetic field gradients) which are temporally different and spatially inhomogeneous. The MR signal used for image reconstruction is recorded as a voltage which is induced in the RF coil arrangement surrounding the examination volume, under the influence of a suitable sequence of magnetic field gradient pulses and RF pulses in the time domain. The actual image reconstruction then takes place by Fourier transformation of the time signals. The sampling of the local frequency space (so-called “k-space”) by means of which the field of view to be imaged and the image resolution are determined is defined by the number, the temporal spacing, the duration and the strength of the magnetic field gradient pulses and RF pulses used. The number of phase encoding steps in the sampling of the k-space and hence the duration of the imaging sequence is predefined by requirements placed on image size and image resolution. This directly results in one of the significant disadvantages of MR imaging, since the recording of an image of the complete examination volume at a resolution sufficient for diagnostic purposes usually takes an undesirably long time.

A large number of technical developments in the field of MR imaging are aimed at drastically shortening the times taken to record an image. Developments in terms of apparatus, which allow as rapid a switching as possible of the magnetic field gradients, have today reached the limits of technical feasibility and the limits of what is physiologically reasonable for the patients. However, for a large number of applications the recording times are still too long.

It seems to have become feasible to overcome the existing technical and physiological limits in terms of the speed of conventional MR imaging by virtue of the parallel methods that have recently become known, such as so-called sensitivity encoding. These methods are based on the knowledge that the spatial sensitivity profile of the RF coil arrangement used stamps a location information item on the MR signal, which location information item can be used for image reconstruction. By virtue of the parallel use of a number of separate coil elements each having different spatial sensitivity profiles, by combining the MR signals recorded in each case it is possible for the time taken to record an image, compared to conventional methods, to be reduced by a factor that in the most favorable case is equal to the number of coil elements used.

A disadvantage of the above-described imaging method, however, is that the RF coil arrangements used therein have coil elements with fixedly predefined spatial sensitivity profiles. Depending on the application, a special arrangement of the coil elements must be selected in order for example that the patient's body part to be examined can be optimally imaged. It is thus a disadvantage of the known parallel MR imaging methods that they cannot be used in a very flexible manner.

More recently, it has been preferred to use in MR devices RF coil arrangements having a number of coil elements that can be actuated individually, namely both for the receive mode and the transmit mode. In such MR devices, it is advantageously possible for the RF field distribution in the examination volume to be fully controllable when generating RF pulses. It is thus possible to generate any conceivable current distribution in the RF coil arrangement by individually setting amplitude and phase within the individual coil elements. By means of the software of the MR device, the RF field distribution within the examination volume can be controlled directly and interactively (so-called “RF shimming”). It is also conceivable to integrate fully automatic control of the RE field homogeneity into the imaging sequence in order to compensate for variable influences on the RF field distribution, for example on account of the different dielectric properties of the patients examined.

Based on this prior art, it is an object of the present invention to provide a universal MR imaging method, by means of which an MR device of the type described above can be used for the particularly rapid recording of images. In particular, the physiological exposure of the patient brought about by the rapid switching of magnetic field gradients and by the generation of RF pulses is to be kept as low as possible.

This object is achieved, based on an MR method of the type mentioned in the introduction, in that the spatial RF field distribution during excitation of the MR signals in method step a) and/or the spatial sensitivity profile of the RF coil arrangement during recording of the MR signals in method step b) are varied by means of the RF coil arrangement.

According to the invention, the RF coil arrangement of the MR device used is designed such that the RF field distribution can be varied during generation of the RF pulses. Thus, for example, gradients of varying degree can be generated in the high frequency field in various spatial directions. By presetting spatially and temporally variable RF field patterns, a location encoding can be stamped on the magnetization distribution excited thereby in the examination volume, it being possible for said location encoding to be used for rapid volume imaging.

It is known that a phase encoding that can be used for image reconstruction can be produced by generating RF field gradients during the excitation of the MR signals (cf. D. I. Hoult: “Rotating Frame Zeugmatography” in “Journal of Magnetic Resonance”, Vol. 33, pages 183 to 197, 1979). In this way, magnetic field gradient pulses used in conventional MR methods for phase encoding can be saved, as a result of which the physiological exposure of the patient is drastically reduced.

Unlike in the method described in the abovementioned article by Hoult et al., according to the invention the phase encoding necessary for image reconstruction takes place by varying the spatial RF field distribution during excitation of the MR signals. By contrast, in conventional imaging using RF field gradients the duration of the RF pulses must be varied in order to obtain the desired phase encoding. By means of the method according to the invention, there is thus also an improvement in terms of the physiological exposure of the patient to high frequency radiation.

By virtue of the varying, according to the invention, of the spatial sensitivity profile of the RF coil arrangement during recording of the MR signals, a location encoding is stamped on the recorded signals, which location encoding can be used in image reconstruction. By contrast with the above-described known parallel imaging methods, according to the invention the MR signals are not recorded by means of a number of different coil elements each having different spatial sensitivity profiles. Rather, the spatial sensitivity profile of the RF coil arrangement used to record the MR signals is varied over time, so that a location encoding that can be predefined depending on the application is stamped on the MR signal recorded at a specific point in time by the spatial sensitivity profile that is active at said point in time. This results in an imaging flexibility that is considerably improved with respect to the methods known from the prior art, since the spatial sensitivity profiles can be adapted individually to the desired examination situation. In terms of the imaging speed that can be achieved, the method according to the invention is comparable with known parallel MR imaging methods.

According to the invention, the spatial sensitivity profile of the RF coil arrangement can expediently be varied during recording of the MR signals by switching the RF coil arrangenent in method step b) between various resonance modes each having different spatial sensitivity profiles. It is readily possible to equip the RF coil arrangement with suitable switchable components, such as PIN diodes or capacitance diodes for example, so that the RF coil arrangement is at a specific predefined resonant frequency depending on the actuation of these components in different resonance modes.

As an alternative, there is the possibility that the MR signals are recorded in method step b) in parallel by means of separate coil elements of the RF coil arrangement, where the spatial sensitivity profile of the RF coil arrangement is varied by the amplitudes and/or the phases of the MR signals recorded by the respective coil elements being varied as a function of time. For this purpose, the MR device used must have a receiving unit which has a number of receiving channels for the individual coil elements of the RF coil arrangement. The receiving unit must have suitable means for varying the amplitudes or the phases of the signals recorded via the individual receiving channels.

An MR device comprising a main field coil for generating a homogeneous, static magnetic field in an examination volume, an RF coil arrangement for generating RF pulses in the examination volume and for recording MR signals from the examination volume, where the RF coil arrangement has a number of coil elements that can be actuated individually to generate the RF pulses and/or to record the MR signals, and comprising a control unit for actuating the RF coil arrangement and also comprising a reconstruction and visualization unit for processing and displaying the MR signals is suitable for carrying out the method according to the invention. The above-described method can be carried out on the MR device according to the invention by means of a suitable program control of the control unit.

In order to vary the spatial RF field distribution during excitation of the MR signals or to vary the spatial sensitivity profile of the RF coil arrangement during recording of the MR signals, the coil elements of the RF coil arrangement in the MR device according to the invention may be designed as inductively coupled loops, where the loops have capacitance diodes or PIN diodes which can be actuated by the control unit of the MR device. By actuating the diodes, the RF coil arrangement can be switched between different resonance modes each having different spatial sensitivity profiles.

The method according to the invention may be made available to users of MR devices in the form of an appropriate computer program. The computer program may be stored on suitable data carriers, such as CD ROMs or disks for example, or may be downloaded from the Internet onto the control unit of the MR device.

The invention will be further described with reference to examples of embodiments shown in the drawings to which, however, the invention is not restricted.

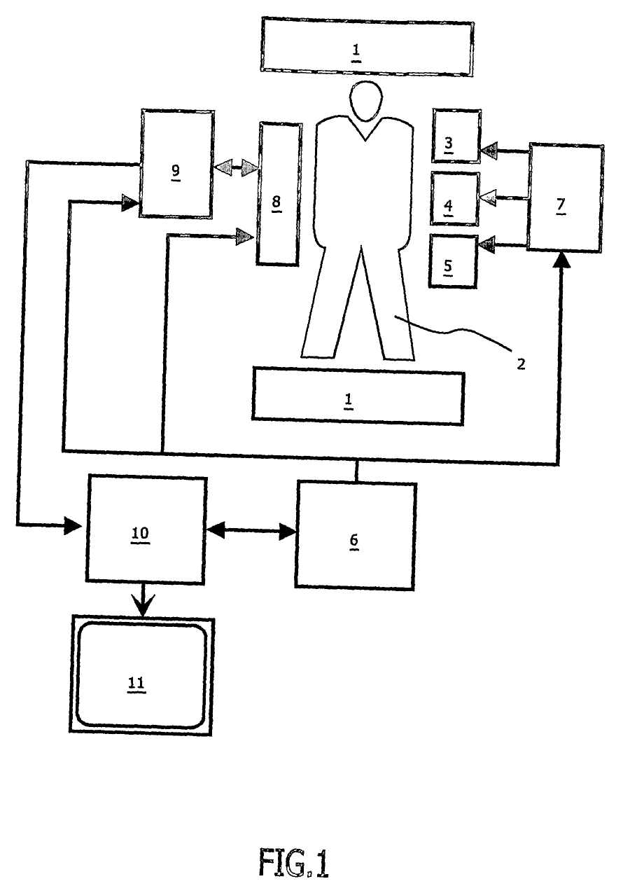

FIG. 1 shows an MR device according to the invention.

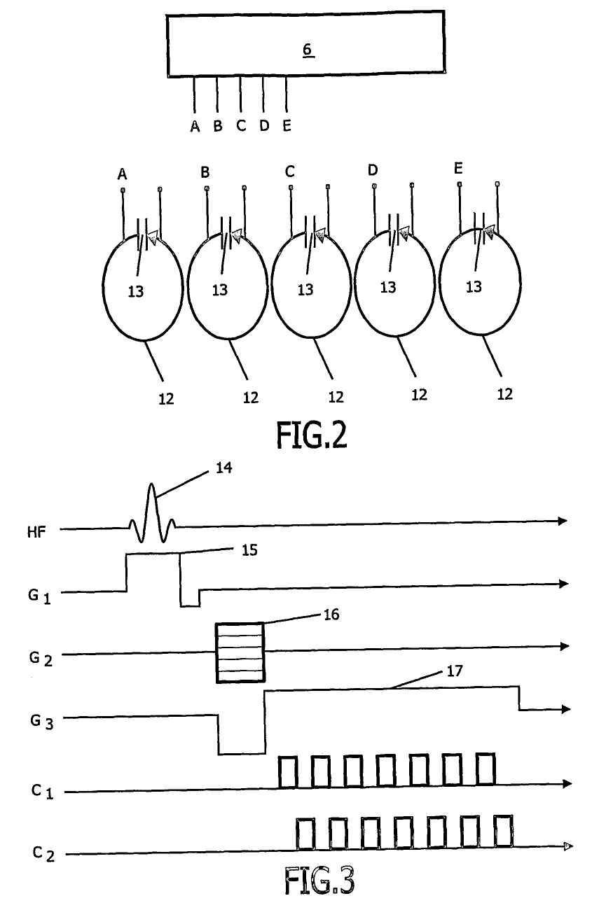

FIG. 2 shows an RF coil arrangement for the MR device shown in FIG. 1.

FIG. 3 shows a pulse sequence of an MR imaging method according to the invention.

FIG. 1 shows an MR device as a block diagram. The device consists of a main field coil 1 for generating a homogeneous, static magnetic field in an examination volume in which a patient 2 is located. The MR device furthermore has gradient coils 3, 4 and 5 for generating magnetic field gradient pulses in different spatial directions within the examination volume. The temporal profile of the magnetic field gradients within the examination volume is controlled by means of a central control unit 6 which is connected to the gradient coils 3, 4 and 5 via a gradient amplifier 7. The MR device shown further includes an RF coil arrangement 8 for generating RF pulses in the examination volume and for recording MR signals from the examination volume. The RF coil arrangement 8 is connected to the control unit 6 and to a reconstruction and visualization unit 10 via a transmitting/receiving unit 9. The MR signals processed by the reconstruction and visualization unit 10 may be displayed by a screen 11. The control unit 6 is also directly connected to the RF coil arrangement 8 in order that the spatial RF field distribution can be varied during the generation of the RF pulses according to the invention. Moreover, the control unit 6 predefines the spatial sensitivity profile of the RF coil arrangement 8 during the recording of the MR signals.

FIG. 2 shows the RF coil arrangement 8 of the MR device in more detail. The RF coil arrangement 8 consists of a number of inductively coupled loops 12, where the resonant response of the overall arrangement is determined by a capacitance diode 13 provided in each loop 12. Each of the capacitance diodes 13 is actuated by the control unit 6 via a corresponding connection A to E, so that the RF coil arrangement 8 shown can be switched between different resonance modes having different spatial sensitivity profiles during recording of the MR signals. If it is desired that according to the invention the spatial RF field distribution is also varied during generation of the RF pulses, that is to say in transmit mode, then for this purpose switchable fixed capacitors may be provided for example in the RF coil arrangement 8 instead of the capacitance diodes, said switchable fixed capacitors being actuated by the control unit 6. The switching between different resonance modes then takes place by means of the switchable fixed capacitors, which have different capacitances depending on the switching state.

The MR method according to the invention is illustrated with reference to the imaging sequence shown schematically in FIG. 3. The sequence begins with the irradiation of an RF pulse 14 by means of which nuclear magnetization in the examination volume is excited. At the same time as the irradiation of the RF pulse 14, a magnetic field gradient pulse 15 is generated which is used to select a slice in the examination volume. There then follows a further magnetic field gradient pulse 16 for phase encoding of the MR signals. After the pulse 16, the MR signals are recorded, namely under the effect of a further magnetic field gradient pulse 17 used for frequency encoding. During the recording of the MR signals, control signals C₁ and C₂ are alternately generated, by means of which the RF coil arrangement is switched back and forth between different resonance modes each having different spatial sensitivity profiles. As a result, temporally successive, alternating MR signals are recorded, which are stamped with a location encoding by the different spatial sensitivity profiles of the RF coil arrangement in the two switching states. This location encoding is then used when the recorded MR signals are combined during image reconstruction.

1. An MR method for generating an image of a body part of a patient located in the examination volume of an MR device, comprising the following method steps:
a) excitation of MR signals in the examination volume by means of a sequence of magnetic field gradient pulses or RF pulses,
b) recording of the MR signals by means of an RF coil arrangement of the MR device,
c) image reconstruction from the recorded MR signals,
wherein the spatial RF field distribution during excitation of the MR signals in method step a) and/or the spatial sensitivity profile of the RF coil arrangement during recording of the MR signals in method step b) are varied by means of the RF coil arrangement.

2. An MR method as claimed in claim 1, wherein during the excitation of the MR signals in method step a), RF field gradients are generated in different spatial directions.

3. An MR method as claimed in claim 1, wherein MR signals recorded in method step b) with different spatial sensitivity profiles of the RF coil arrangement are combined in the image reconstruction in method step c).

4. An MR method as claimed in claim 1, wherein the RF coil arrangement is switched between various resonance modes (C₁, C₂) in method step a) and/or in method step b).

5. An MR method as claimed in claim 1, wherein the MR signals are recorded in method step b) in parallel by means of separate coil elements of the RF coil arrangement, where the spatial sensitivity profile of the RF coil arrangement is varied by the amplitudes and/or the phases of the MR signals recorded by the respective coil elements being varied.

6. An MR device comprising a main field coil generating a homogeneous, static magnetic field in an examination volume, an RF coil arrangement for generating RF pulses in the examination volume and for recording MR signals from the examination volume, where the RF coil arrangement has a number of coil elements that can be actuated individually to generate the RF pulses and/or to record the MR signals, and comprising a control unit for actuating the RF coil arrangement and also comprising a reconstruction and visualization unit for processing and displaying the MR signals, wherein the control unit has a program control which can be used to carry out a method as claimed in claim 1 on the MR device.

7. An MR device as claimed in claim 6, wherein coil elements of the RF coil arrangement are designed as inductively coupled loops, where the loops have capacitance diodes, PIN diodes or switchable fixed capacitors which can be actuated by the control unit of the MR device.

8. A computer program for an MR device as claimed in claim 6, wherein the computer program can be used to implement a method as claimed in claim 1 on the control unit of the MR device.
